(19) United States
(12) Patent Application Publication
(10) Pub. No.: US 2006/0112503 A1
(43) Pub. Date: Jun. 1, 2006

(54) Solid personal care composition

(75) Inventors: Satoru Hatano, Ashiya (JP); Virginia Ann Lam, Baltimore, MD (US)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 11/289,058

(22) Filed: Nov. 29, 2005

Related U.S. Application Data
(60) Provisional application No. 60/631,385, filed on Nov. 29, 2004.

(51) Int. Cl. A61K 8/81; A61K 8/00

(52) U.S. Cl. 8/406; 424/61; 424/63

(57) ABSTRACT

Disclosed is a personal care composition comprising: (a) at least a solid hydrophobic component; and (b) no more than 25% by weight of the composition of a soap component, and the composition having: (i) a needle penetration, as measured according to the ASTM Test Method D5, of from about 1 to about 40 at 25° C.; (ii) a yield stress of at least about 1500 Pa at 25° C.; and (iii) a viscosity of between about 1 mPas and about 10,000,000 mPas at 100° C.

### CROSS REFERENCE TO RELATED APPLICATION

This application claims the benefit of U.S. Provisional Application No. 60/631,385, filed on Nov. 29, 2004.

### TECHNICAL FIELD

The present invention relates to a personal care composition which is suitable for application to a target personal area when heated. The composition provides a solid film on the target area which, based on the characteristics of the composition, provides unique benefits of the composition.

### BACKGROUND

Mascara products are used to enhance the beauty of a person's eyes by coating the eyelashes to primarily thicken, lengthen, color, and define the individual eyelashes. For the last 60 years, mascara products have been provided in the form of mascara applicators having an applicator brush attached to a handle, the applicator brush portion dipped in a package, such as a tube, containing a liquid to semi-solid mascara composition. Mascara compositions typically take the form of emulsions or dispersions of waxes and pigments in water or other volatile carriers. Mascara composition and delivery systems are limited by emulsion or solution chemistry and film forming technologies that are applied wet and then dried to create a film of mascara that sets and holds the eyelashes.

These mascara compositions which are liquid to semi-solid have a low viscosity profile and low yield point, such that they are inherently prone to smearing and smudging after application to the eyelashes. The solid components dispersed in the composition, such as waxes, may also be difficult to apply, as clumping and globbing may occur due to lack of film smoothness of the solid components.

From another aspect, solvents and carriers of the composition that do not evaporate in timely manner may also provide smearing and smudging after application to the eyelashes. The so-called waterproof mascaras intend to solve such problem by employing volatile hydrocarbon solvents. While such volatile hydrocarbon solvents provide wear benefits, the application and beauty benefits may be compromised. Further, the volatile hydrocarbon solvents may cause odor and safety concerns.

One solution for providing a mascara composition having improved application and improved wearability, is to provide the composition solid, wherein the composition is heated prior to application to the eyelashes, for softening, and/or smoothing the composition upon application. Such heating would also benefit in shortening the evaporation time required after application. The solid film provided on the eyelashes after application would have a much higher yield point than films made by conventional mascara compositions, thereby being less prone to smearing and smudging.

Meanwhile, solid personal care composition heated upon use for application may also provide other benefits based on the characteristics of the composition.

Based on the foregoing, there is a need for a personal care composition which is solid at room temperature, and suitable for application to a target personal area when heated. None of the existing art provides all of the advantages and benefits of the present invention.

### SUMMARY OF THE INVENTION

The present is directed to a personal care composition comprising

a) at least a solid hydrophobic component; and

b) no more than 25% by weight of the composition of a soap component; the composition having:
i) a needle penetration, as measured according to the ASTM Test Method D5, of from about 1 to about 40 at 25° C.;
ii) a yield stress of at least about 1500 Pa at 25° C.; and
iii) a viscosity of between about 1 mPas and about 10,000,000 mPas at 100° C.

The present composition is applied to a target personal area by softening or liquefying the composition via heat, and applying the composition to the target personal area. The present composition is so designed to provide suitable physicochemical properties for the usage when heated to a predetermined temperature.

These and other features, aspects, and advantages of the present invention will become evident to those skilled in the art from a reading of the present disclosure with the appended claims.

### DETAILED DESCRIPTION

While the description concludes with claims particularly pointing out and distinctly claiming the invention, it is believed that the present invention will be better understood from the following description.

All percentages, parts and ratios are based upon the total weight of the compositions of the present invention, unless otherwise specified. All such weights as they pertain to listed ingredients are based on the active level and, therefore do not include carriers or by-products that may be included in commercially available materials.

All ingredients such as actives and other ingredients useful herein may be categorized or described by their cosmetic and/or therapeutic benefit or their postulated mode of action. However, it is to be understood that the active and other ingredients useful herein can, in some instances, provide more than one cosmetic and/or therapeutic benefit or operate via more than one mode of action. Therefore, classifications herein are made for the sake of convenience and are not intended to limit an ingredient to the particularly stated application or applications listed.

Personal Care Composition

The composition of the present invention may be used for various usages in the personal care field, particularly for application on keratinous tissues. Unlimited examples of such usages include cosmetic usage and treatment of eyelashes and eyebrows; treatment, styling, coloring, and removal of hair; treatment and tattooing of skin; and nail coloring. The compositions herein are used with heating devices having suitable arrangements for the specific use.

The present composition is applied to a target personal area by softening or liquefying the composition via heat, and applying the composition to the target personal area. The present composition is so designed to provide suitable physicochemical properties for the usage when heated to a predetermined temperature.

For example, the composition may be a cosmetic, tattooing, hair styling, hair coloring, or nail coloring composition which is softened at a predetermined temperature for application to the target personal area; such as facial skin, eyelashes, eyebrows, nails, or hair; which is then left to cool to form a solid film. The composition thus applied to the target personal area provides a firm film covering the desired area, which is not softened at body temperature, and thus provides enhanced wearability, coloring, or styling. These compositions may further comprise a pigment, dye, or a film forming agent.

In another example, the composition may be a skin treatment or hair treatment composition which is softened at a predetermined temperature at which penetration and/or effectiveness of a certain component is enhanced upon application to the target personal area; such as skin, hair or scalp; and also warming the same area. The component may be the essential solid hydrophobic component alone, or other components. Other components which may be further comprised are skin or hair active agents.

One highly preferred embodiment of the present composition is a mascara composition. The softened state of the mascara composition is fluid enough to be applied to the eyelashes with an average number of strokes of an applicator holding the composition, however, is viscous enough to stay on the applicator upon application, and on the eyelashes after application. The mascara composition thus applied to the eyelashes provides a firm film, which is not softened at body temperature, and thus provides enhanced wearability. Eyelash curling devices, such as those described in WO 99/22782, may be employed as an applicator providing ample heat.

The personal care composition of the present invention is solid at room temperature, namely 25° C., and is softened with elevated temperature such that it can be applied to the eyelashes. By “solid” herein for describing the personal care composition, what is meant is that the composition has a certain hardness to retain its structure, and also that the composition is stable against stress or shear. The present composition has a needle penetration, as measured according to the American Standard prescribed by the American Society for Testing and Materials (ASTM) Test Method D5, of from about 1 to about 40, and a yield stress of no less than about 1500 Pa, both measurements at 25° C., preferably at 35° C.

The principle of the measurement of the needle penetration according to the ASTM D5 consists of measuring the depth, expressed in tenths of a millimeter, to which a standard needle (weighing 2.5 g and placed in a needle holder weighing 47.5 g, i.e., a total of 50 g) penetrates when placed on the composition for 5 seconds. The principle of the measurement of yield stress consists of measuring oscillation stress sweep for understanding flow behavior and viscoelastic character for fluids and semi-solids as a function of stress, shear rate, or temperature. In the present invention, the yield stress is measured using a TA Instrument Rheometer AR-500 using a 40 mm Al Parallel Plate (Gap: 600 mm) at 1 Hz. At 25° C., the present composition preferably has no measurable yield point under stress or shear.

The personal care composition changes rheology as it is heated, and finally reaches a point where it is liquid by 100° C., preferably by 90° C. By “liquid” herein for describing the personal care composition, what is meant is that the composition has a viscosity of between about 1 mPas and about 10,000,000 mPas. The present personal care composition goes through a transition change in terms of rheology between 25° C. and 100° C., such that during these temperatures, there is a range of temperature and rheology in which the composition is suitably softened or liquefied for application to the target personal area. The softened state of the personal care composition is fluid enough to be applied to the target personal area with the expected method of application of the composition. Cosmetic, tattoing, hair styling, hair coloring, nail coloring compositions are designed so that a noticeable amount of composition stays on the skin, nails, or hair. These compositions are designed so that, at the determined elevated temperature, the composition still exhibits enough viscosity to be held on the applicator to be used for application, and thereby a sufficient amount of composition can be applied to the target personal area. For mascara and hair styling compositions, the elevated temperature band may be selected to provide suitable balance of applicability of the personal care composition and eyelash or hair curling/lifting or styling benefit. Typically, the temperature band is between about 50° C. and about 100° C.

The components for the composition are selected in order to provide the desired rheology profile. The composition comprises at least a solid hydrophobic component for providing the essential physical characteristics of the present invention. The composition may be made solely by the solid hydrophobic component.

Another essential feature of the present composition, however, is that the composition is not one which is applied to the target personal area via the use of water for wetting and deforming the composition. Namely, the present composition is distinguished from mascara cakes or soaps known in the art, such as in U.S. Pat. No. 5,053,220. The present composition comprises no more than 25% by weight of the composition of a soap component.

The composition may take the phase form of an oil mixture, the oil being mainly made by a solid hydrophobic component, or a water-in-oil emulsion further comprising an emulsifier and water. Water-in-oil emulsion forms are suitable for encompassing water-soluble or water-dispersible components.

Solid Hydrophobic Component

The present composition comprises a solid hydrophobic component for providing the solid characteristic of the personal care composition. Solid hydrophobic components are typically used at levels from about 25% to about 100% in oil mixture forms, and from about 25% to about 95% in water-in-oil emulsion forms. Suitable solid hydrophobic components include waxes and fats.

Waxes are defined as lower-melting organic mixtures or compounds of high molecular weight, solid at room temperature and generally similar in composition to fats and oils except that they contain no glycerides. Some are hydrocarbons, others are esters of fatty acids and alcohols. Waxes useful in the present invention are selected from the group consisting of animal waxes, vegetable waxes, mineral waxes, synthetic waxes petroleum waxes, ethylenic polymers, hydrocarbon types such as Fischer-Tropsch waxes, silicone waxes, and mixtures thereof wherein the waxes have a melting point between 25° C. and 100° C.

The specific waxes useful in the present invention are selected from the group consisting of beeswax, lanolin wax, shellac wax (animal waxes); carnauba, candelilla, bayberry (vegetable waxes); ozokerite, ceresin, (mineral waxes); paraffin, microcrystalline waxes (petroleum waxes); polyethylene, (ethylenic polymers); polyethylene homopolymers (Fischer-Tropsch waxes); C24-45 alkyl methicones (silicone waxes); and mixtures thereof.

Highly preferable commercially available waxes herein include stearyl palmitate by the tradename PURESTER 34, available from Strahl & Pitsch, ceresin by the tradename CERESIN 252 available from Strahl & Pitsch, and paraffin wax by the tradenames PARAFFIN SP-673P, PARAFFIN 206, and PARAFFIN 192 available from Strahl & Pitsch.

Useful herein are fats, namely glyceryl esters of higher fatty acids such as stearic and palmitic. Such esters and their mixtures are solid at room temperature and exhibit crystalline structure. The fats employed according to the invention are selected from the group consisting of fats derived from animals, vegetables, synthetically derived fats, and mixtures thereof wherein said fats have a melting point from about 55° C. to about 100° C. Preferably, the fats are selected from the group consisting of glyceryl monostearate, glyceryl distearate, glyceryl tristearate, palmitate esters of glycerol, C18-36 triglycerides, glyceryl tribehenate, C18-36 acid triglycerides and mixtures thereof.

Highly preferable commercially available fats herein include glyceryl monostearate by the tradename CUTINA GMS-V available from Cognis Cutina.

Soap Component

The present composition comprises no more than 25% by weight of the composition of a soap component. By having a controlled amount of a soap component, the present composition is not deformed via the use of water for wetting. The present composition may be completely devoid of soap components, or comprise a small amount of soap component for ease of cleaning off the present composition upon washing with water.

By “soap component” herein, what is meant is are compounds having cleaning activity composition a negatively charged carboxylate group and a cation selected from metal ions, earth metal ions, amines, and others. Soap components herein include sodium, potassium, magnesium, and triethanol amine salts of saturated and unsaturated, straight and branched chain fatty acids having from about 4 to about 36 carbon atoms. Non-limiting examples of soap components herein include sodium salts of saturated straight chain fatty acids of from about 14 to about 22 carbon atoms, potassium salts of the same fatty acids, triethanol amine stearate, and others.

Emulsifiers

The compositions of the present invention in emulsion form comprises an emulsifier, which is typically a lipophilic surfactant, preferably by weight of the entire composition at from about 1% to about 15%. The lipophilic surfactant herein has an HLB value of less than about 8.

The HLB value is a theoretical index value which describes the hydrophilicity-hydrophobicity balance of a specific compound. Generally, it is recognized that the HLB index ranges from 0 (very hydrophobic) to 40 (very hydrophilic). The HLB value of the lipophilic surfactants may be found in tables and charts known in the art, or may be calculated with the following general equation: HLB=7+(hydrophobic group values)+(hydrophilic group values). The HLB and methods for calculating the HLB of a compound are explained in detail in Surfactant Science Series, Vol. 1: Nonionic Surfactants”, pp 606-13, M. J. Schick (Marcel Dekker Inc., New York, 1966).

Without being bound by theory, the species and levels of the lipophilic surfactant herein are believed to provide a stable water-in-oil emulsion in view of the other components of the present invention.

The lipophilic surfactant can be an ester-type surfactant. Ester-type surfactants useful herein include: sorbitan monoisostearate, sorbitan diisostearate, sorbitan sesquiisostearate, sorbitan monooleate, sorbitan dioleate, sorbitan sesquioleate, glyceryl monoisostearate, glyceryl diiostearate, glyceryl sesquiisostearate, glyceryl monooleate, glyceryl dioleate, glyceryl sesquioleate, diglyceryl diisostearate, diglyceryl dioleate, diglycerin monoisostearyl ether, diglycerin diisostearyl ether, and mixtures thereof.

Commercially available ester-type surfactants are, for example, sorbitan isostearate having a tradename Crill 6 available from Croda, and sorbitan sesquioleate with tradename Arlacel 83 available from Kao Atras.

The lipophilic surfactant can be a silicone-type surfactant. Silicone-type surfactants useful herein are (i), (ii), and (iii) as shown below, and mixtures thereof.
(i) dimethicone copolyols having the formulation: 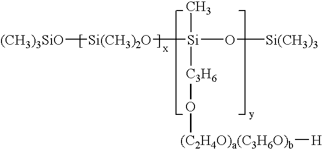
   wherein x is an integer from 5 to 100, y is an integer from 1 to 50, a is zero or greater, b is zero or greater, the average sum of a+b being 1-100.
(ii) dimethicone copolyols having the formulation: 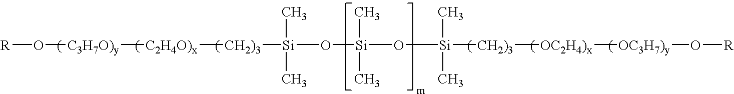
   wherein R is selected from the group consisting of hydrogen, methyl, and combinations thereof, m is an integer from 5 to 100, x is independently zero or greater, y is independently zero or greater, the sum of x+y being 1-100.
(iii) branched polyether-polydiorganosiloxane emulsifiers herein having the formulation: 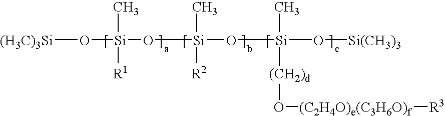
   wherein R¹ is an alkyl group having from about 1 to about 20 carbons; R² is 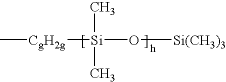
   wherein g is from about 1 to about 5, and h is from about 5 to about 20; R³ is H or an alkyl group having from about 1 to about 5 carbons; e is from about 5 to about 20; f is from about 0 to about 10; a is from about 20 to about 100; b is from about 1 to about 15; c is from about 1 to about 15; and d is from about 1 to about 5.

Commercially available silicone-type surfactants are, for example, dimethicone copolyols DC5225C, BY22-012, BY22-008, SH3746M, SH3771M, SH3772M, SH3773M, SH3775M, SH3748, SH3749, and DC5200, all available from Dow Corning, and branched polyether-polydiorganosiloxane emulsifiers such as PEG-9 polydimethylsiloxyethyl Dimethicone, having an HLB of about 4 and a molecular weight of about 6,000 having a tradename KF 6028 available from ShinEtsu Chemical.

Water

The composition of the present invention in water-in-oil form comprises water in an amount sufficient to provide a discontinuous aqueous phase, preferably an amount such that water is no more than about 50%, more preferably from about 10% to about 40% of the entire composition. Use of water allows the inclusion of useful components such as film forming polymers which are hydrophilic and/or aqueous carrier-based, hydrophilic conditioning agents, and other water soluble or water dispersible components described below.

In the present invention, deionized water is typically used. Water from natural sources including mineral cations can also be used, depending on the desired characteristic of the product.

Pigments and Dyes

The compositions of the present invention may comprise pigments and dyes selected from the group consisting of inorganic pigments, organic pigments, and organic lake pigments, pearlescent pigments, laked tar color dyes, laked natural color dyes, and mixtures thereof.

When employed, the pigments and dyes are present in proportions depending on the color and the intensity of the color that it is intended to produce. When employed, the level of pigments and dyes in the composition is from about 3% to about 25%, preferably from about 5% to about 15%. The pigments may optionally be surface-treated with treatments that include, but are not limited to, silicones, perfluorinated compounds, lecithin, and amino acids.

Inorganic pigments useful in the present invention include those selected from the group consisting of rutile titanium dioxide, anatase titanium dioxide (both coded in the Color Index under the reference CI 77891); black, yellow and red iron oxides (CI 77499, 77492 and 77491); bismuth oxychloride (CI 77163); manganese violet (CI 77742); ultramarines (CI 77007); chromium oxide (CI 77288); chromium hydroxide (CI 77289); ferric ferrocyanide (CI 77510); zinc oxide (CI 77947); and mixtures thereof.

The organic pigments useful in the present invention include carbons black, and the dyes and the analogous lakes selected from the group consisting of D&C Red 6 (CI 15850); D&C Red 7 (CI 15850:1); D&C Red 21 (CI 45380:2); D&C Red 22 (CI 45380); D&C Red 27 (CI 45410:1); D&C Red 28 (CI 45410); D&C Red 30 (CI 73360); D&C Red 33 (CI 17200); D&C Red 34 (CI 15880:1); D&C Red 36 (CI 12085); D&C Orange 4 (CI 15510); D&C Orange 5 (CI 45370:1); D&C Orange 11 (CI 45425); FD&C Yellow 5 (CI 19140), FD&C Yellow 6 (CI 15985); D&C Yellow 10 (CI 47005); FD&C Green 3 (CI 42053); D&C Green 5 (CI 61570); FD&C Blue 1 (CI 42090); Cochineal Carmine (CI 75470); Guanine (CI 75170) and mixtures thereof.

The pearlescent pigments useful in the present invention include those selected from the group consisting of mica (or a similar plate-like substrate) coated with any of the following materials alone or in combination: titanium dioxide, bismuth oxychloride, iron oxides, ferric ferrocyanide, chromium oxide, chromium hydroxide, and any organic pigment of the above-mentioned type and mixtures thereof.

Film Forming Polymer

The compositions of the present invention may comprise a film forming polymer, for imparting styling, wear, or transfer resistant properties. When included, such materials are typically used in an amount of from about 0.5% to about 20% preferably from about 0.5% to about 10% by weight of the composition. Preferred polymers form a non-tacky film which is removable with water used with cleansers such as soap and shampoo. The film forming polymers herein can be hydrophobic or hydrophilic, and can be provided in a lipophilic or aqueous carrier. When polymers provided in aqueous carriers are employed in the composition, a water-in-oil form is selected. Polymers of hydrophilic nature are also compatible with a water-in-oil form composition.

Examples of suitable film forming polymeric materials include:
a) sulfopolyester resins, such as those with tradename AQ sulfopolyester resins, such as AQ29D, AQ35S, AQ38D, AQ38S, AQ48S, and AQ55S available from Eastman Chemicals;
b) polyvinylacetate/polyvinyl alcohol polymers, such as tradename Vinex resins available from Air Products, including Vinex 2034, Vinex 2144, and Vinex 2019;
c) acrylic resins, including water dispersible acrylic resins available from National Starch under the trade name “Dermacryl”, including Dermacryl LT;
d) acrylates and their derivative polymers, including acrylates copolymer with tradename Luvimer available from BASF, Avalure series available from Noveon, Daitosol 5000AD available from Daito Kasei Kogyo, ethylene.styrene/acrylates copolymer such as Syntran series available from Interpolymer, acrylates/ammonium methacrylate copolymer with tradename Ultrasol 2075C available from Presperse, octyl acrylates copolymer with tradename Daitotol SJ available from Kobo, acrylates silicone copolymer with tradename Daitotol ASC available from Kobo, AMP-acrylates/allyl methacrylate copolymer with tradename Fixate G100 Polymer available from Noveon, acrylate/dimethicone copolymer with tradename KP545 available from ShinEtsu;
e) styrene, such as sodium polystyrene sulfonate with tradename Flexan available from National Starch;
f) urethanes, such as polyurethane-1 polymer with tradename Luviset PUR available from BASF;
g) polyvinylpyrrolidones (PVP), including tradenames Luviskol K17, K30 and K90 available from BASF PVP K-30, PVP K-120 available from ISP, tricontanyl PVP with tradename Ganex WP 660 Resin available from ISP, water soluble copolymers of PVP, including PVP/VA S-630 and W-735 and PVP/dimethylaminoethylmethacrylate Copolymers such as Copolymer 845, Copolymer 937, and Styleze CC-10 available from ISP, VP/DAM available from Daiichi Kogyo Seiyaku, PVP/acrylates/lauryl methacrylate copolymer with tradename Styleze 2000 available from ISP;
h) high molecular weight silicones such as dimethicone and organic-substituted dimethicones, especially those with viscosities of greater than about 50,000 mPas;
i) high molecular weight hydrocarbon polymers with viscosities of greater than about 50,000 mPas such as polybutene, polybutene terephthalate, polydecene, polycyclopentadiene, and similar linear and branched high molecular weight hydrocarbons, including isooctane with tradename Permethyl 97A available from Presperse;
j) organosiloxanes, including organosiloxane resins, fluid diorganopolysiloxane polymers and silicone ester waxes.

Also useful herein are latex polymers including copolymer PVP/hexadecane or the copolymer PVP/eicosene marketed by ISP under the tradenames Ganex V-216® and Ganex V-220®, respectively. Ganex V-216® is a PVP/hexadecane copolymer comprising approximately 15-23% of pyrrolidone units with a weight average molecular weight of 7300. Ganex V-220® is a copolymer PVP/eicosene which comprises approximately 20-28% of pyrrolidone units and a weight average molecular weight of 8600.

Skin and Hair Active Agents

The compositions of the present invention may further comprise a skin active agent or a hair active agent. When used, the skin and hair active agents herein are used in a safe and effective amount, by which is meant an amount high enough to deliver the desired skin, scalp, or hair benefit at the predetermined elevated temperature, but low enough to avoid serious side effects, at a reasonable benefit to risk ratio within the scope of sound medical judgement. Skin and hair active agents herein include their pharmaceutically-acceptable salts, by which is meant any of the commonly-used salts that are suitable for use in contact with human tissues without undue toxicity, irritation, incompatibility, allergic response, and the like.

Anti-Acne Actives: Anti-acne actives can be effective in treating and preventing acne vulgaris, a chronic disorder of the pilosebaceous follicles. Preferred anti-acne actives include benzoyl peroxide, lactic acid, 4-methoxysalicylic acid, metronidazole, niacinamide, panthenol, retinoic acid and derivatives thereof, salicylic acid, sulphur, triclosan, zinc oxide, and mixtures thereof.

Artificial Tanning Agents and Accelerators: Artificial tanning agents can help in simulating a natural suntan by increasing melanin in the skin or by producing the appearance of increased melanin in the skin. Non-limiting examples of artificial tanning agents and accelerators include dihydroxyacetone, glucose tyrosinate and acetyl tyrosine, brazilin, caffeine, coffee extracts, DNA fragments, isobutyl methyl xanthine, methyl xanthine, PHOTOTAN (available from Laboratoires Serobiologiques located in Somerville, N.J.), prostaglandins, tea extracts, theophylline, UNIPERTAN P2002 (available from Unichem, located in Chicago, Ill.) and UNIPERTAN P27 (available from Unichem, located in Chicago, Ill.); and mixtures thereof.

Antiseptics: Suitable antiseptics for use herein include alcohols, benzoate, sorbic acid, and mixtures thereof.

Anti-microbial and Anti-fungal Actives: Anti-microbial and anti-fungal actives can be effective to prevent the proliferation and growth of bacteria and fungi. Non-limiting examples of antimicrobial and antifungal actives include ketoconazole, benzoyl peroxide, tetracycline, benzalkonium chloride, benzoic acid and its salts, butyl paraben, cinnamon oil, citronella oil, echinacea, ethyl paraben, GLYDANT PLUS (available from Lonza located in Fairlawn, N.J.), grapefruit seed oil, iodopropynl butyl carbamide lemon balm oil, salicylic acid, sodium metabisulphite, sodium sulphite, sorbic acid and its salts, and tea tree oil.

Skin Soothing Agents: Skin soothing agents can be effective in preventing or treating inflammation of the skin. The soothing agent enhances the skin appearance benefits of the present invention, e.g., such agents contribute to a more uniform and acceptable skin tone or colour. Examples of skin soothing agents include allantoin, aloe, bisabolol, borage oil, chamomile, evening primrose, panthenol, and tocopherol.

Sunscreening Agents: Sunscreens useful herein include both inorganic sunscreens such as titanium and zinc oxides, as well as the many commercially available UVA and UVB absorbing organic sunscreens.

Skin Barrier Repair Aids: Skin barrier repair actives are those skin care actives which can help repair and replenish the natural moisture barrier function of the epidermis. Non-limiting examples of skin barrier repair aids include ceramides, cholesterol, lanolin, lanolin alcohols, n-acetyl cysteine, n-acetyl-L-serine, niacinamide, nicotinic acid and its esters, nicotinyl alcohol, panthenol, phosphodiesterase inhibitors, trimethyl glycine, tocopheryl nicotinate, and vitamin D3 and analogues or derivatives.

Anti-Wrinkle and Anti-Skin Atrophy Actives: Anti-wrinkle and anti-skin atrophy actives can be effective in replenishing or rejuvenating the epidermal and/or dermal layer. These actives generally provide these desirable skin care benefits by promoting or maintaining the natural process of desquamation and/or building skin matrix components (e.g., collagen and glycosaminoglycans). Examples of antiwrinkle and anti-skin atrophy actives include niacinamide, nicotinic acid and its esters, nicotinyl alcohol, estrogens and estrogenic compounds, or mixtures thereof.

Skin Repair Actives: Skin repair actives can be effective in repairing the epidermal and/or dermal layer. Non-limiting examples of skin repair actives include adenosine, aloe derived lectins, ascorbyl palmitate, azaleic acid, biotin, blackberry bark extract, catecholamines, chalcones, cis retinoic acid, citric acid esters, coenzyme Q10 (ubiquinone), dehydrocholesterol, dehydroepiandrosterone, dehydroascorbic acid and derivatives thereof, dehydroepiandrosterone sulphate, estrogen and its derivatives, farnesol, gingko bilboa extracts, ginseng extracts, lactate dehydrogenase inhibitors, magnesium ascorbyl phosphate, melatonin, N-acetyl cysteine, pantethine, phytic acid and its salts, retinal, retinol, retinyl acetate, retinyl propionate and vitamin K.

Skin Lightening Agents: Skin lightening agents can actually decrease the amount of melanin in the skin or provide such an effect by other mechanisms. Skin lightening agents suitable for use herein include arbutin, ascorbic acid, ascorbyl palmitate, azelaic acid, butyl hydroxy anisole, gallic acid and its derivatives, glycyrrhizinic acid, hydroquinine, inositol ascorbate, kojic acid, n-acetyl glucosamine, niacinamide and vitamin B₃ and its analogues.

Sebum Inhibitors: Sebum inhibitors can decrease the production of sebum in the sebaceous glands. Examples of suitable sebum inhibitors include dichlorophenyl imidazoldioxolan, aluminium hydroxy chloride, corticosteroids and cucumber extracts.

Sebum Stimulators: Sebum stimulators can increase the production of sebum by the sebaceous glands. Non-limiting examples of sebum stimulators include bryonolic acid, dehydroepiandrosterone and orizanol.

Sensates: Non-limiting examples of suitable sensates for use herein include agents which impart a cool feel such as camphor, thymol, 1-menthol and derivatives thereof, eucalyptus, carboxamides; menthane ethers and menthane esters; and agents imparting a warm feel such as cayenne tincture, cayenne extract, cayenne powder, vanillylamide nonanoate, nicotinic acid derivatives (benzyl nicotinate, methyl nicotinate, phenyl nicotinate, etc.), capsaicin, nasturtium officinale extract, Zanthoxylum piperitum extract and ginger extract, or mixtures thereof.

Protease Inhibitors: Protease inhibitors are compounds which inhibit the process of proteolysis, that is, the splitting of proteins into smaller peptide fractions and amino acids. Examples of suitable protease inhibitors include A E COMPLEX (available from Barnet Products located in Englewood, N.J.), BLUE ALGAE EXTRACT (available from Collaborative Labs Inc. located in East Setauket, N.Y.), and SEPICONTROL AS (available from Seppic located in Paris, France).

Skin Tightening Agents: Examples of skin tightening agents include sodium polystyrene sulphonate, BIOCARE SA (available from Amerchol located in Edison, N.J.) and egg albumen.

Anti-Itch Ingredients: Examples of anti-itch ingredients include ichthyol and OXYGENATED GLYCERYL TRIESTERS (available from Laboratoires Seporgia located in Sophia Antipolis, France.)

Hair Growth Inhibitors: Suitable agents for inhibiting hair growth include 17 beta estradiol, anti angiogenic steroids, curcuma extract, cycloxygenase inhibitors, evening primrose oil, linoleic acid and 5-alpha reductase inhibitors such as ethynylestradiol and, genistine.

Desquamation Enzyme Enhancers: These agents enhance the activity of endogenous desquamating enzymes. Non-limiting examples of desquamation enzyme enhancers include N-methyl serine, serine, trimethyl glycine, and mixtures thereof.

Anti-Glycation Agents: Anti-glycation agents prevent the sugar induced crosslinking of collagen. A suitable example of an anti-glycation agent includes AMADORINE (available from Barnet Products Distributor located in Englewood, N.J.).

Depilatory Agents: Depilatory agents decompose keratin structure for ease of removal. Non-liming examples of depilatory agents include thioglycolate salts such as sodium thioglycolate, potassium thioglycolate, and sodium salicylate.

Hydrophobic Conditioning Agents

The compositions of the present invention may further comprise a hydrophobic conditioning agent. Nonlimiting examples of hydrophobic conditioning agents include those selected from the group consisting of mineral oil, petrolatum, lecithin, hydrogenated lecithin, lanolin, lanolin derivatives, C7-C40 branched chain hydrocarbons, C1-C30 alcohol esters of C1-C30 carboxylic acids, C1-C30 alcohol esters of C2-C30 dicarboxylic acids, monoglycerides of C1-C30 carboxylic acids, diglycerides of C1-C30 carboxylic acids, triglycerides of C1-C30 carboxylic acids, ethylene glycol monoesters of C1-C30 carboxylic acids, ethylene glycol diesters of C1-C30 carboxylic acids, propylene glycol monoesters of C1-C30 carboxylic acids, propylene glycol diesters of C1-C30 carboxylic acids, C1-C30 carboxylic acid monoesters and polyesters of sugars, polydialkylsiloxanes, polydiarylsiloxanes, polyalkarylsiloxanes, cyclomethicones having 3 to 9 silicon atoms, polysiloxane crosspolymers such as vinyl dimethicone crosspolymer available as a dimethicone mixture fluid with tradename KSG series available from ShinEtsu, vegetable oils, hydrogenated vegetable oils, polypropylene glycol C4-C20 alkyl ethers, di C8-C30 alkyl ethers, and combinations thereof.

Hydrophobic Conditioning Agents

The compositions of the present invention may further comprise a hydrophilic conditioning agent. Nonlimiting examples of hydrophilic conditioning agents include those selected from the group consisting of polyhydric alcohols, polypropylene glycols, polyethylene glycols, ureas, pyrolidone carboxylic acids, ethoxylated and/or propoxylated C3-C6 diols and triols, alpha-hydroxy C2-C6 carboxylic acids, ethoxylated and/or propoxylated sugars, polyacrylic acid copolymers, sugars having up to about 12 carbons atoms, sugar alcohols having up to about 12 carbon atoms, and mixtures thereof.

Solvents

The compositions of the present invention may contain a volatile or non-volatile solvent that dissolves or uniformly disperses certain components of the present invention. They include, but are not limited to, lower alcohols (such as ethanol, isopropanol), dihydric alcohols such as propylene and butylene glycol, polyols such as glycerin, hydroalcoholic mixtures, hydrocarbons (such as isobutane, hexane, decene, acetone), halogenated hydrocarbons (like Freon), linalool, hydrocarbon esters (such as ethyl acetate, dibutyl phthalate), volatile silicon derivatives, especially siloxanes (such as phenyl pentamethyl disiloxane, phenethyl pentamethyl disiloxane, methoxypropyl heptamethyl cyclotetrasiloxane, chloropropyl pentamethyl disiloxane, hydroxypropyl pentamethyl disiloxane, octamethyl cyclotetrasiloxane, decamethyl cyclopentasiloxane), and mixtures thereof.

Additional Components

The compositions hereof may further contain additional components such as are conventionally used in personal care products, e.g., for providing aesthetic or functional benefit to the composition, hair, or skin, such as sensory benefits relating to appearance, smell, or feel, therapeutic benefits, or prophylactic benefits (it is to be understood that the above-described required materials may themselves provide such benefits).

The CTFA Cosmetic Ingredient Handbook, Second Edition (1992) describes a wide variety of nonlimiting cosmetic and pharmaceutical ingredients commonly used in the industry, which are suitable for use in the topical compositions of the present invention. Such other materials may be dissolved or dispersed in the composition, depending on the relative solubilities of the components of the composition.

Examples of suitable topical ingredient classes include: sunscreen actives, anti-cellulite agents, antioxidants, radical scavengers, chelating agents, vitamins and derivatives thereof, abrasives, other oil absorbents, astringents, dyes, essential oils, fragrance, structuring agents, emulsifiers, solubilizing agents, anti-caking agents, antifoaming agents, binders, buffering agents, bulking agents, denaturants, pH adjusters, propellants, reducing agents, sequestrants, cosmetic biocides, and preservatives, such as propylparaben, methyl paraben, phenoxyethanol, benzyl alcohol, and EDTA and its salts.

### EXAMPLES

The following examples further describe and demonstrate the preferred embodiments within the scope of the present invention. The examples are given solely for the purpose of illustration, and are not to be construed as limitations of the present invention since many variations thereof are possible without departing from its spirit and scope.

### Examples 1-3

Examples 1 and 2 are mascara compositions in the form of oil mixture, and Example 3 is a mascara composition in the form of a water-in-oil emulsion. Each composition has a needle penetration of between 1-25 at 35° C., a yield stress of over 1500 Pa at 35° C., and has a viscosity of between 1 mPas and 10,000,000 mPas at 90° C. The mascara compositions can be suitably applied to the eyelashes when heated to between 50-80° C.

Composition

Following components are used at the respective w/w %.

| No. | Component | 1 | 2 | 3 |
|---|---|---|---|---|
| 1 | Glyceryl Monostearate-Vegetable | 8.00 | 10.00 | 10.00 |
| | Derived *1 | | | |
| 2 | Stearyl Palmitate *2 | 26.00 | 18.00 | 17.00 |
| 3 | Ceresine Wax *3 | 26.00 | 18.00 | 17.00 |
| 4 | Paraffin Wax *4 | 15.50 | 9.70 | 7.87 |
| 5 | Dimethicone/Vinyl Dimethicone | | 25.00 | |
| | Crosspolymer *5 | | | |
| 6 | Tricontanyl PVP *6 | 2.00 | 2.00 | 2.00 |
| 7 | Sorbitan Sesquioleate *7 | | | 5.00 |
| 8 | Propylparaben | 0.30 | 0.30 | 0.10 |
| 9 | CI 77499 (iron oxide) and | 22.20 | 17.00 | 15.00 |
| | Methicone *8 | | | |
| 10 | Deionized Water | | | 20.00 |
| 11 | Sodium Polystyrene Sulfonate *9 | | | 5.00 |
| 12 | Phenoxyethanol | | | 0.30 |
| 13 | Methylparaben | | | 0.20 |
| 14 | Benzyl alcohol | | | 0.40 |
| 15 | Trisodium EDTA | | | 0.13 |

Definition of components
*1 Glyceryl Monostearate-Vegetable Derived: GMS-V available from Cognis
*2 Stearyl Palmitate: Purester 34 available from Strahl & Pitsch
*3 Ceresine Wax: Ceresine Wax SP-252 available from Strahl & Pitsch
*4 Paraffin Wax: Paraffin Wax SP-673P available from Strahl & Pitsch
*5 Dimethicone/Vinyl Dimethicone Crosspolymer: KSG 16 available from ShinEtsu
*6 Tricontanyl PVP: Ganex WP-660 available from ISP
*7 Sorbitan Sesquioleate: Crill 6 available from Croda
*8 CI 77499 (iron oxide) and Methicone: Si Black Iron Oxide available from Daito Kasei
*9 Sodium Polystyrene Sulfonate: Flexan II available from National Starch & Chemical

Method of Preparation

Examples 1-3 may be made in any suitable method known to one skilled in the art. Preferably, the examples are made by the following methods.

### Example 1

1) Heat components 1-4 to 85-90° C. Being low shear mixing when enough wax has melted.
2) Once components 1-4 have completely melted, add components 6 and 8. Continue mixing for about 10 min.
3) Add component 9 to product of Step 2, and disperse for 30 min-1 hr with a dispersator.
4) Pour the product of Step 3 into molds and allow product to cool and solidify.

### Example 2

1) Heat components 1-4 to 85-90° C. Being low shear mixing when enough wax has melted.
2) Once components 1-4 has completely melted, add component 5. Continue mixing for about 10 min.
3) Add components 6 and 8 to product of Step 2. Continue mixing for about 10 min.
4) Add component 9 to product of Step 3, and disperse for 30 min-1 hr with a dispersator.
5) Pour the product of Step 4 into molds and allow product to cool and solidify.

### Example 3

1) Heat components 1-4 to 85-90° C. Being low shear mixing when enough wax has melted.
2) Separately heat components 10-15 to 85-90° C. and mix with low shear mixing.
3) Once components 1-4 has completely melted, add components 6-8. Continue mixing for about 10 min.
4) Add component 9 to product of Step 3, and disperse for 30 min-1 hr with a dispersator 2. Homogenize for 30 min-1 hr by mixing.
5) Add the product of Step 2 to product of Step 4 while mixing with low shear mixing.
6) Further mix product of Step 5 with moderate shear mixing for 15-30 min. to effect emulsification.
7) Pour the product of Step 6 into molds and allow product to cool and solidify.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A personal care composition comprising:
a) at least a solid hydrophobic component; and
b) no more than 25% by weight of the composition of a soap component; the composition having:
i) a needle penetration, as measured according to the ASTM Test Method D5, of from about 1 to about 40 at 25° C.;
ii) a yield stress of at least about 1500 Pa at 25° C.; and
iii) a viscosity of between about 1 mPas and about 10,000,000 mPas at 100° C.

2. The composition according to claim 1 in the form of an oil mixture.

3. The composition according to claim 1 in the form of a water-in-oil emulsion further comprising water and an emulsifier.

4. The composition according to any of claim 1 wherein the composition is for cosmetic, tattooing, hair styling, hair coloring, or nail coloring.

5. The composition of claim 4 further comprising a pigment or dye.

6. The composition of claim 4 further comprising a film forming agent.

7. The composition according to any of claim 1 wherein the composition is for skin treatment, hair treatment, skin removal, or hair removal.

8. The composition of claim 4 further comprising a skin or hair active agent.
